(19) United States
(12) Patent Application Publication
(10) Pub. No.: US 2016/0151412 A1
(43) Pub. Date: Jun. 2, 2016

(54) COMBINATION OF XENON WITH AN NMDA RECEPTOR ANTAGONIST FOR FIGHTING A NEURODEGENERATIVE DISEASE

(71) Applicants: L'AIR LIQUIDE, SOCIETE ANONYME POUR L'ETUDE ET L'EXPLOITATION DES PROCEDES GEORGES CLAUDE, Paris (FR); L'INSTITUT DU CERVEAU ET DE LA MOELLE EPINIERE, Paris (FR)

(72) Inventors: Patrick MICHEL, Paris (FR); Jérémie LAVAUR, Paris (FR); Etienne HIRSCH, Versailles (FR); Marc LEMAIRE, Paris (FR); Jan PYPE, Herne (BE)

(21) Appl. No.: 14/903,809

(22) PCT Filed: Jul. 3, 2014
(86) PCT No.: PCT/FR2014/051710

§ 371 Date: Jan. 8, 2016

(30) Foreign Application Priority Data
Jul. 8, 2013 (FR) 1356688

(51) Int. Cl. A61K 33/00; A61K 45/06; A61K 9/00

(52) U.S. Cl. CPC A61K 33/00; A61K 9/007; A61K 45/06

(57) ABSTRACT

The invention relates to a gaseous medication containing xenon, preferably in an amount of less than 75% by volume, for use by inhalation, in combination with at least one NMDA receptor antagonist in the form of a liquid or solid, in particular memantine, for treating, slowing or preventing neurological deterioration consequent upon a neurodegenerative disease, in particular Alzheimer's disease, in a human patient.

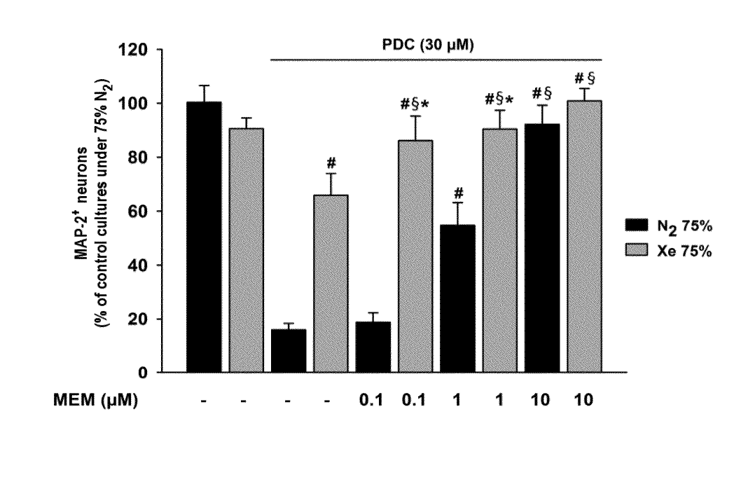

### CROSS REFERENCE TO RELATED APPLICATIONS

This application is a 371 of International PCT Application PCT/FR2014/051710, filed Jul. 3, 2014, which claims priority to French Patent Application No. 1356688, filed Jul. 8, 2013, the entire contents of which are incorporated herein by reference.

### BACKGROUND

The invention relates to the use of xenon gas as an inhalable medicine, used in combination with an NMDA receptor antagonist, namely memantine or nitromemantine, to treat, slow or prevent a neurological damage associated with or resulting from a neurodegenerative disease, in particular Alzheimer's disease or Parkinson's disease.

N-Methyl-D-aspartate (NMDA) receptors/channels are molecular entities of the plasma membrane of neuronal cells. These receptors are the target of glutamate molecules released in the synaptic and extrasynaptic space, glutamate being an excitatory neurotransmitter that provides communication from one nerve cell to another.

In neurodegenerative diseases such as Alzheimer's disease, nerve cells produce glutamate and release it in abnormally high amounts, thus causing neuronal excitotoxicity due to excessive stimulation of NMDA receptors. This excitotoxicity, suspected to be a party to Alzheimer's disease (Hynd et al., Neurochem Int, 2004) and also to other degenerative pathologies such as Parkinson's disease (Mehta et al., Eur J Pharmacol, 2013), has harmful consequences for postsynaptic neurons bearing NMDA receptors, notably following a too-great influx of calcium ions (Ca²⁺) into the intracellular compartment.

Memantine, a non-competitive low-affinity NMDA receptor antagonist, is used to fight Alzheimer's disease. Memantine interacts with NMDA receptors directly, by blocking them, and by limiting the influx of Ca²⁺ ions, thus reducing toxicity due to excess glutamate. The result is an improved transmission of nerve signals between neuronal cells and a slowing of the decline of memory and cognition in the context of Alzheimer's disease.

However, the effect of memantine is limited and this molecule is not stripped of undesirable effects, such as confusion, dizziness, sleepiness, headaches, insomnia, agitation, hallucinations, vomiting, anxiety, etc.

The problem is thus to propose an improved treatment that avoids, slows or minimizes any neurological damage associated with or resulting from a neurodegenerative disease of the Alzheimer's disease or Parkinson's disease type, while decreasing the doses of memantine used and thus the negative undesirable effects associated with the use of this compound.

### SUMMARY

The solution, according to the present invention, is a gaseous medicine containing xenon for use by inhalation, in combination with at least one NMDA receptor antagonist in liquid or solid form, to treat, slow or prevent a neurological damage resulting from a neurodegenerative disease in a human patient.

Indeed, in the context of the present invention, it was shown that the combination of xenon and an NMDA receptor antagonist, such as memantine or a derivative or compound of memantine, in particular nitromemantine, produces a synergistic action by these compounds and that such a combination can constitute a promising treatment for neurological damage resulting from neurodegenerative diseases, in particular such as Alzheimer's disease.

Such a combination rests in particular on the modes of action of these compounds.

Thus, xenon inhibits excitatory glutamate signaling pathways via its antagonistic action on NMDA receptors, but also on α-amino-3-hydroxy-5-methylisoazol-4-propionate (AMPA) receptors, as well as on kainate receptors, which make up ionotropic glutamate receptors.

Consequently, the combination xenon/memantine or xenon/nitromemantine produces a synergy of action at glutamate and in particular NMDA receptors, without risking an increase in the undesirable effects of memantine.

In other words, adding xenon strengthens the beneficial effects of memantine or nitromemantine by a synergistic effect but without causing the undesirable effects of memantine or nitromemantine.

### BRIEF DESCRIPTION OF THE DRAWING

For a further understanding of the nature and objects for the present invention, reference should be made to the following detailed description, taken in conjunction with the accompanying drawing, wherein the sole FIGURE illustrates a chart showing the impact of Memantine and Xenon on -trans-pyrrolidine-2,4-dicarboxylic acid induced neural degeneration in ex vivo primary cortex neurons.

### DESCRIPTION OF PREFERRED EMBODIMENTS

As the case may be, the gaseous medicine according to the invention can comprise one or more of the following features:
the NMDA receptor antagonist is in solid form, in particular tablet or capsule form.
the NMDA receptor antagonist is memantine, or a derivative or compound of memantine.
the NMDA receptor antagonist is memantine or nitromemantine.
the neurodegenerative disease is Alzheimer's disease or Parkinson's disease, preferably Alzheimer's disease.
the daily dose of NMDA receptor antagonist administered to the patient is less than or equal to 20 mg/day.
the xenon gas is administered to the patient before, during or after administration of the NMDA receptor antagonist, preferably after administration of the NMDA receptor antagonist.
the neurological damage comprises an excessive influx of Ca²⁺ ions in one or more populations of vulnerable neurons of the patient.
it further contains oxygen, preferably at least 21% oxygen by volume.
it consists of xenon and oxygen.
it consists of xenon, nitrogen and oxygen.
the patient is a man or a woman.
the patient is a human being over 30 years of age, preferably over 40 years of age, in particular over 50 years of age.
the medicine contains an effective amount of xenon.
the medicine contains a non-anesthetic, i.e., sub-anesthetic, amount of xenon.
it contains between 10% and 80% xenon by volume.
the medicine contains between 15% and 80% xenon by volume.
the medicine contains at least 20% xenon by volume.
the medicine contains less than 60% xenon by volume.
the medicine less than 75% xenon by volume.
the medicine contains between 20% and 50% xenon by volume.
the medicine contains between 20% and 40% xenon by volume.
the xenon is mixed with the oxygen just before or at the time it is inhaled by the patient or is provided in the form of a “ready-to-use” gas mixture premixed with oxygen, and optionally contains another gas compound, for example nitrogen.
the medicine consists of a gas mixture formed of oxygen and nitrogen.
the gas is administered to the patient one or more times per day.
the gas is administered to the patient for a period of inhalation of a few minutes to a few hours, typically between 15 minutes and 6 hours, preferably fewer than 4 hours.
the period, dosing schedule and frequency of administration are a function of the change in the neurological state of the patient concerned and will preferably be set by the doctor or medical staff according to the neurological state of the patient concerned.
the gaseous medicine is packaged in a gas cylinder having a capacity (water equivalent) up to 50 liters, typically of the order of 0.5 to 15 liters, and/or at a pressure less than or equal to 350 bar absolute, typically a pressure between 2 and 300 bar.
the gaseous medicine is packaged in a gas cylinder equipped with a valve or an integrated valve/regulator for controlling the flow rate and optionally the pressure of the gas delivered.
the gaseous medicine is packaged in a gas cylinder made of steel, aluminum or composite materials.
during the treatment, the gaseous medicine is administered to the patient by inhalation by means of a facial or nasal mask, nasal cannula or through any other system for administering an inhalable gas.

In other words, the invention concerns an inhalable medicinal combination or association comprising xenon gas and at least one NMDA receptor antagonist in liquid or solid form for use to treat, slow or prevent a neurological damage resulting from a neurodegenerative disease in a human patient.

According to another aspect, the invention also relates to a therapeutic treatment method to treat, slow or prevent at least one neurological damage resulting from a neurodegenerative disease in a human patient, wherein:
i) a human patient having a neurodegenerative disease or likely to have such a neurodegenerative disease is identified,
ii) a gaseous medicine containing xenon is administered to the patient via inhalation, and
iii) at least one NMDA receptor antagonist in liquid or solid form is administered to said patient,
   so as to provide neuronal protection and thus to treat, slow or prevent at least one neurological damage resulting from the neurodegenerative disease in said patient.

Preferably, in step i):
the human patient is a man or a woman.
the human patient is over 30 years of age, preferably over 40 years of age, in particular over 50 years of age.
the patient is identified by a doctor or similar.
the patient is identified by thorough screening.
the neurodegenerative disease likely to cause at least one neurological damage is Alzheimer's disease or Parkinson's disease, preferably Alzheimer's disease.
said neurological damage comprises an excessive influx of Ca²⁺ ions in one or more populations of vulnerable neurons of the patient.

Preferably, in step ii):
at least one NMDA receptor antagonist in solid form is administered to said patient.
at least one NMDA receptor antagonist is administered enterally, i.e., orally.
at least one NMDA receptor antagonist in tablet or capsule form is administered to said patient.
memantine, or a derivative or compound of memantine as NMDA receptor antagonist is administered to the patient.
memantine or nitromemantine as NMDA receptor antagonist is administered to the patient.
a daily dose of NMDA receptor antagonist of less than or equal to 20 mg/day is administered to the patient.
at least one NMDA receptor antagonist is administered to said patient before, during or after the inhalation of xenon by the patient.

Preferably, in step iii):
the period, dosing schedule and frequency of administration of xenon are selected and/or set as a function of the change in the neurological state of the patient concerned.
an effective amount of xenon is administered.
a non-anesthetic amount of xenon is administered.
from 10% to 80% xenon by volume, preferably between 20% and 50% xenon by volume, is administered.
the xenon is mixed with the oxygen before or at the time it is inhaled by the patient, preferably with at least 21% oxygen by volume.
a ready-to-use gaseous mixture consisting of xenon and oxygen (binary mixture) or of xenon, oxygen and nitrogen (ternary mixture) is administered.
the xenon gas is administered to the patient one or more times per day.
the xenon gas is administered to the patient for a period of inhalation of a few minutes to a few hours, typically between 15 minutes and 6 hours, preferably fewer than 4 hours.
the xenon gas is administered by means of a facial or nasal mask, nasal cannula or through any other system or device for administering gas to a patient.

### Example

In order to demonstrate the efficacy of the combination of xenon and an NMDA receptor antagonist according to the present invention, we set up a cell model of cortical neurons in which neuronal death is triggered by blocking glutamate reuptake systems by means of extended treatment with L-trans-pyrrolidine-2,4-dicarboxylic acid (PDC), a compound whose mechanism of action has previously been disclosed by Zuiderwijk et al., (Europ J Pharmacol, 1996).

The technique implemented is disclosed below and the results obtained are illustrated in the appended FIGURE showing the synergistic protective effects of xenon and memantine in a cell model mimicking chronic cortical degeneration.

### Protocol for Obtaining Primary Cultures of Cortex

Cultures are prepared from rat embryo cortex taken from Wistar rates, on day 15.5 of gestation.

The method for obtaining cortex cultures comprises the preparation of a homogeneous cell suspension by mechanical, i.e., non-enzymatic, dissociation of embryonic tissue using Leibovitz's L15 medium (Sigma Aldrich).

Aliquots of this suspension are added to Nunc 48-well plates coated beforehand with a thin layer of polyethylenimine (1 mg/ml, borate buffer, pH 8.3) to allow neuronal cells to adhere (see Toulorge et al., Faseb J, 2011).

The seeding density is between about 20,000 and 30,000 cells/cm².

The cortex cultures are maintained in Neurobasal culture medium containing an oxidant-free B27 cocktail, N2 supplement, glutamine (2 mM) and a penicillin/streptomycin cocktail (see Nafia et al., J Neurochem, 2008). The medium and the supplement are available from Life Technologies.

Six hours after placing in culture, 0.5 μM of the antimitotic cytarabine (Sigma Aldrich) is added to limit the proliferation of astrocytes.

Until the moment when the effects of the gases of interest are evaluated, the cultures are placed in a conventional enclosure thermostated at 37° C., wherein CO₂ is maintained at 5% by volume and the atmosphere is saturated with water.

The culture medium is not changed during the culture period.

### Pharmacological Treatments of the Cultures

The degenerative process is triggered by applying a glutamate reuptake system blocker, PDC (Zuiderwijk et al., Europ J Pharmacol, 1994).

The NMDA receptor blocker, memantine, is added to the cultures before the application of PDC. These two products (PDC and memantine) are from RD Systems.

### Maintaining the Cultures Under Controlled Gaseous Atmosphere

Once the pharmacological treatments are carried out, the multi-well plates containing the cells in culture and the plate used for humidification of the internal compartment of the chamber are placed on a metal base that receives the Plexiglas incubation chamber. The two parts (base and Plexiglas chamber) are screwed together in a joined manner.

A gas mixture of interest comprising (% by volume): 20% O₂, 5% CO₂ and 75% of the gas tested is then injected into the incubation chamber, with inlet and outlet valves open, while controlling the outlet flow by means of a flowmeter. The gases tested are nitrogen and xenon.

The reference outlet flow, set for air at 10 liters/minute, is corrected as a function of the density of the mixture used.

When the CO₂ measurement at the outlet reaches 5%, the injection of the gas mixture is stopped and the chamber is completely sealed by closing the inlet and outlet valves.

The exposure chamber is then placed in a 37° C. enclosure throughout the experimental protocol.

### Immunodetection of Microtubule-Associated Protein-2 (MAP-2) and Cell Counts

After breaking the seal by opening the inlet and outlet valves and unscrewing the chamber from its base, the cultures are fixed with 4% formaldehyde in PBS for 12 minutes and then incubated at 4° C. with a monoclonal anti-MAP-2 antibody (dilution 1:200; clone AP-20; Sigma-Aldrich) for 3 days.

This antibody is detected with an anti-mouse secondary antibody coupled with cyanine-3 (Sigma Aldrich; dilution 1:1000 in PBS).

Image capturing is carried out with the 10× objective using a Nikon TE-300 inverted microscope equipped with an ORCA-ER cooled digital camera and HCl image capturing software (Hamamatsu).

The results obtained in the model of cortical degeneration using chronic PDC treatment reveal synergistic protective effects between xenon and memantine, which are summarized in the following Table and are shown in the FIGURE.

TABLE
Summary of the principal results of the in vitro study on cortex cultures
Day-16 cortex cultures

| | Gas mixture | |
|---|---|---|
| | (20% O₂ + 5% CO₂ + | |
| | 75% gas tested); % by | Neuronal |
| Treatments, days 12-16 | volume | survival |
| Control group I | N₂ | +++ |
| Control group II | Xe | +++ |
| PDC (30 μM) group | N₂ | − |
| PDC (30 μM) group | Xe | ++ |
| PDC (30 μM) + memantine (0.1 μM) | N₂ | − |
| group | | |
| PDC (30 μM) + memantine (0.1 μM) | Xe | +++ |
| group | | |
| PDC (30 μM) + memantine (1 μM) | N₂ | ++ |
| group | | |
| PDC (30 μM) + memantine (1 μM) | Xe | +++ |
| group | | |
| PDC (30 μM) + memantine (10 μM) | N₂ | +++ |
| group | | |
| PDC (30 μM) + memantine (10 μM) | Xe | +++ |
| group | | |

In the preceding Table, a favorable response, synonymous with a decrease in neuronal cell death in the presence of the treatments of interest, is indicated by a “+,” “++,” or “+++” sign (+++=reference level). Conversely, an unfavorable response is represented by a “−” sign, synonymous with an increase in neuronal death.

Memantine acts by blocking NMDA receptors and xenon by blocking these same receptors but likely via a distinct mechanism involving the blocking of the binding site of glycine, an amino acid that acts as a coactivator of the receptor.

Cytarabine, or Ara-C, is first applied to all the culture wells in order to limit the proliferation of astrocytes. As for PDC, it induces a degenerative process by preventing reuptake of the glutamate produced and released endogenously by the neuronal cells in culture.

In view of the results shown in the appended FIGURE, one notes that the combination of xenon and an NMDA receptor antagonist, namely memantine here, produces a neuroprotective effect significantly superior to the effect of each molecule taken separately.

In fact, a genuine synergy of action of the combination xenon/memantine is established.

First, these tests showed that the blocking of glutamate reuptake systems by PDC causes a loss of about 85% of cortical neuronal cells, at a concentration of 30 μM, under an atmosphere containing 75% nitrogen by volume.

However, the deleterious effects of PDC are partially prevented when nitrogen is replaced with xenon as the survival rate increases from 15.9% to 65.9%, respectively.

Interestingly, memantine has no significant effect in this cell model when it is applied alone at 0.1 μM, under atmosphere containing 75% nitrogen. However, it potentiates the protective effects obtained under 75% xenon, making it possible to reach a survival rate superior to 86.1% under these conditions, versus about 65.9% in the absence of memantine.

The potentiation of the effects of xenon is also observed when a 1 μM concentration of memantine is applied, which makes it possible to reach, by itself, a survival rate of 54.6%.

Lastly, at 10 μM memantine, the synergy is no longer observed, given that the memantine itself already exerts a protective effect which saves more than 90% of the neuronal cells.

The results disclosed in the FIGURE appended hereto show the synergistic protective effects of xenon and memantine in a cell model mimicking chronic cortical degeneration.

These results were obtained on rat cortex cultures treated from day 12 in vitro and for the following 4 days with a glutamate reuptake inhibitor, PDC (30 μM), under atmosphere containing 75% nitrogen (N₂ 75) or 75% xenon (Xe 75), in the presence or absence of memantine (MEM), tested at 0.1, 1 or 10 μM.

The cultures are then collected for fixing and analysis. Neuronal survival is quantified in the various experimental conditions tested by counting the number of cell bodies immunopositive for the pan-neuronal marker MAP-2.

The results are expressed in % of cultures not treated with PDC, maintained under 75% nitrogen (control condition).

Thus, the statistical study made by one-way analysis of variance (ANOVA) followed by a Student-Newman-Keuls test (n=6 for each experimental point) shows that:
xenon alone (without memantine) produced an effect significantly superior to that of the nitrogen-alone condition (^{#} p<0.05, increased in relation to the cultures treated with 30 μM PDC under 75% nitrogen).
xenon has a synergistic effect with memantine, significantly superior to the xenon-alone condition (^{§} p<0.05, increased in relation to the cultures treated with 30 μM PDC under 75% xenon) and to the 75% nitrogen condition, even in the presence of memantine at the concentrations of 0.1 and 1 μM (* p<0.05, increased in relation to the cultures treated with 30 μM PDC under 75% nitrogen, in the presence of the same concentration of memantine).

Xenon thus produces, when it is combined with an NMDA receptor antagonist, such as memantine or nitromemantine as NMDA receptor antagonist, a synergistic effect in the treatment, slowing or prevention of neurological damage resulting from a neurodegenerative disease, in particular Alzheimer's disease.

1.-17. (canceled)

18. A method for treating at least one neurological damage resulting from a neurodegenerative disease in a human patient, wherein:
i) the human patient is identified as having a neurodegenerative disease or likely to have the neurodegenerative disease,
ii) a gaseous medicine containing xenon is administered to the patient via inhalation, and
iii) at least one NMDA receptor antagonist in liquid or solid form is further administered to said patient,
so as to provide neuronal protection and thus to treat or slow or the at least one neurological damage resulting from the neurodegenerative disease in said human patient.

19. The method as claimed in claim 18, characterized in that the neurodegenerative disease is Alzheimer's disease or Parkinson's disease.

20. The method as claimed in claim 18, characterized in that the neurological damage comprises an excessive influx of Ca²⁺ ions in one or more populations of vulnerable neurons of the patient.

21. The method as claimed in claim 18, characterized in that the NMDA receptor antagonist is memantine or nitromemantine.

22. The method as claimed in claim 18 wherein the gaseous medicine containing xenon contains between 10% and 80% xenon by volume.

23. The method as claimed in claim 18 wherein the NMDA receptor antagonist is administered in a tablet or capsule form.

24. The method as claimed in claim 18 wherein the daily dose of NMDA receptor antagonist administered to the patient is less than or equal to 20 mg/day.

25. The method as claimed in claim 18 wherein the gaseous medicine containing xenon is administered to the human patient after the step of administration of the NMDA receptor antagonist.

26. The method as claimed in claim 18 wherein gaseous medicine containing xenon further contains oxygen.

27. The method as claimed in claim 26 wherein gaseous medicine containing xenon further contains at least 21% oxygen by volume.

28. The method as claimed in claim 26 wherein gaseous medicine containing xenon contains xenon and oxygen or xenon, oxygen and balance nitrogen.

29. The method as claimed in claim 18 wherein gaseous medicine containing xenon contains at least 20% xenon by volume and/or it contains less than 75% xenon by volume.

30. The method as claimed in claim 18 wherein gaseous medicine containing xenon contains less than 60% xenon by volume.

31. The method as claimed in claim 18 wherein the human patient is over 50 years of age.

32. The method as claimed in claim 18 wherein the gaseous medicine containing xenon is administered to the patient, one or more times per day.

33. The method as claimed in claim 18 wherein the gaseous medicine containing xenon is administered to the patient for a period of inhalation of between 15 minutes and 4 hours.
